(19) United States
(12) Patent Application Publication
(10) Pub. No.: US 2008/0201831 A1
(43) Pub. Date: Aug. 28, 2008

(54) Neck Protective Device

(75) Inventor: Luigi Ronco, Molvena VI (IT)

Correspondence Address: NIXON & VANDERHYE, PC, 901 NORTH GLEBE ROAD, 11TH FLOOR, ARLINGTON, VA, 22203, US

(73) Assignee: DAINESE S.p.A., Molvena VI (IT)

(21) Appl. No.: 11/791,206

(22) PCT Filed: Nov. 22, 2005
(86) PCT No.: PCT/IB05/53866

§ 371 Date: Aug. 17, 2007

(30) Foreign Application Priority Data
Nov. 22, 2004 (IT) RM2004 A 000572
Jul. 1, 2005 (IT) RM2005 A 000350

(51) Int. Cl. A41D 13/00; A61F 5/00

(52) U.S. Cl. 2/468; 602/18

(57) ABSTRACT

A neck protective device, comprising at least two protective elements (100), partially overlapped therebetween, supporting means (200) and respective connecting means (300) of the protective elements (100), and characterized in that the connecting means (300) is apt to allow the mutual rotation of the protective elements (100), working as hinges along different axes of rotation (401, 402), in a manner such that the protective elements (100) slide the one against the other one.

The present invention refers to a neck protective device.

Of late years, attention to body protection during sports activities and, in general, all those situations potentially hazardous when not carried out or occurring under appropriate safety conditions, has remarkably increased.

In sports entailing high-speed situations, such as motor biking, but also skiing, biking, riding, such a need is particularly felt and has led to researching high-resistance protections concomitantly exhibiting high wearability in those body parts that are in motion during such activities and, also for this reason, particularly prone to accidents.

The use of new materials and manufacturing technologies enabled to make ever more effective protective elements enabling sport people, as well as other people, to stand up again, practically unhurt, after spectacular falls.

However, in spite of this effort, the protection of the neck, which, as can easily be imagined, is of vital importance, has often been neglected, or anyhow not sufficiently effective.

In fact, dramatic accidents causing permanent invalidities to sport people, or even killing them, owing to violent knocks or excessive and unnatural motions at the neck, are unfortunately still too frequent.

On the other hand, the neck protection devices existing to date entail drawbacks making them either less than effective in protection or anyhow less than practical in use, and therefore actually not useful in a number of activities.

A first category of protections is essentially made of soft material, enabling to absorb knocks and, somehow, contain neck motions, preventing those particularly hazardous excessive and unnatural motions.

However, these devices do not provide a sufficient protection in case of high-speed impacts, as those occurring in skiing or in motor biking.

On the other hand, to be at least partially effective they should be particularly bulky, the presence of a lot of material being required to absorb knocks at the neck.

Therefore, to provide greater protection to the neck there have been developed protections of rigid material anatomically adjusting to the user's body at the neck.

Such devices are used, e.g., in motorsport disciplines so as to form a sort of harness, protecting the neck from knocks and keeping it blocked in the event of accidents.

In this case, for instance, while an improved protection against knocks is possible, the defense against hyperextensions and unnatural motions of the neck is implemented essentially by inhibiting thereto any motion, but its torsions; therefore, said devices allow no freedom of motion when worn-on.

Such a feature is particularly negative in those activities requiting anyhow ample freedom of motion, even though the protection of the neck is desirable.

Moreover, such protections are fastened to a user at his/her shoulders and such a fastening system limits the motion of the latter as well.

Instead, above all in amateur practice of such particularly hazardous activities, personal safety is surely important, yet a freedom of motion sufficing not to cause excessive discomfort to the user is also desirable.

A compromise between the two solutions described hereto is represented, e.g., by some protections used in American football practice, in which a rigid protective element for the neck is connected to a shoulder-covering harness.

In this case a front tilting of the head is possible, though to a limited extent, ensuring anyhow a high protection.

However, in this case said protective devices prove particularly bulky, and above all do not allow the side tilting of the neck, nor the torsion thereof.

Hence, the technical problem underlying the present invention is to provide a neck protective device overcoming the drawbacks mentioned above with reference to the known art.

Such a problem is solved by a neck protective device according to claim 1.

The present invention provides several relevant advantages. The main advantage lies in that it allows an effective protection of the neck, cushioning impacts as well as limiting hyperextension and unnatural motions, while being compact and assuring freedom of motion, comfort and high wearability.

Other advantages, features and the modes of employ of the present invention will be made apparent from the following detailed description of some embodiments thereof, given by way of non-limiting example. Reference will be made to the figures of the annexed drawings, wherein:

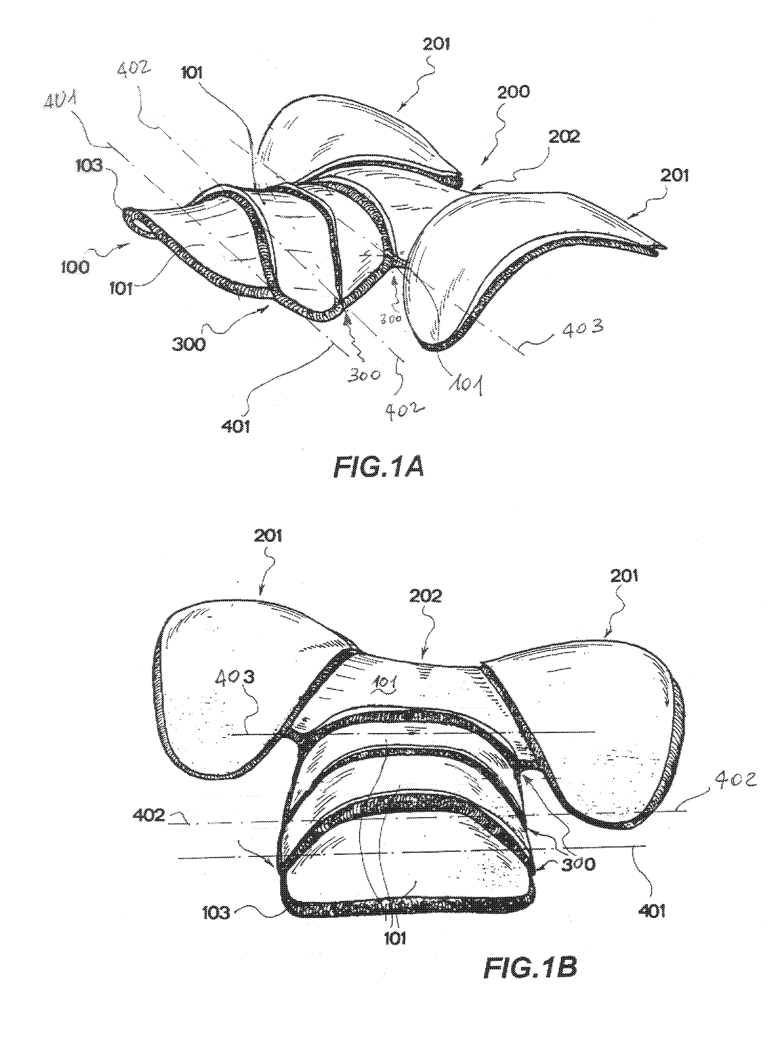

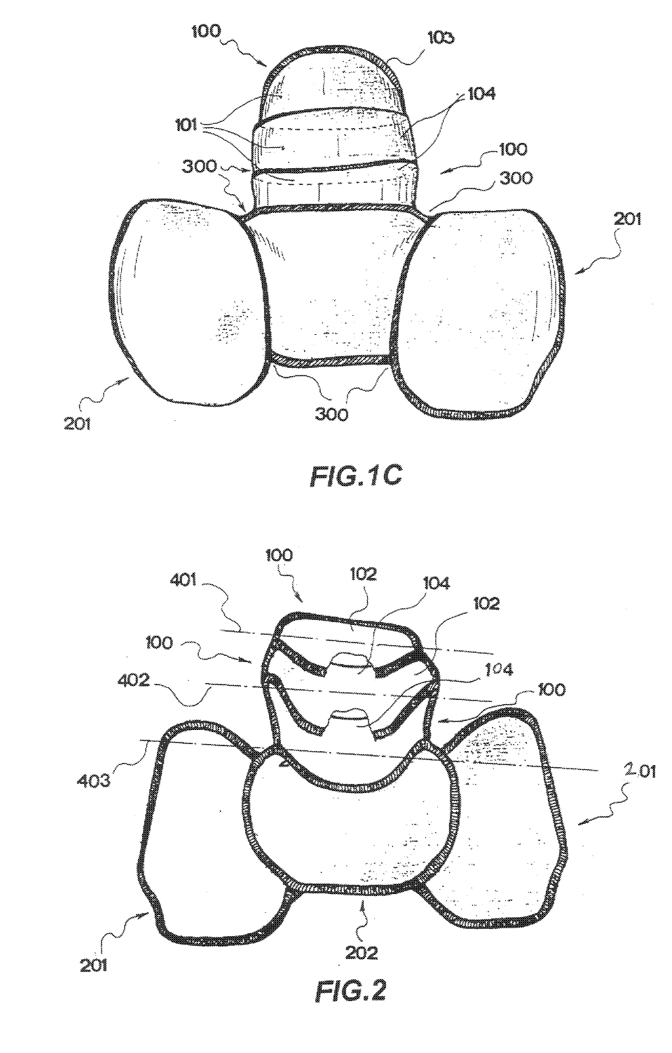

FIGS. 1A to 1C are different perspective views of a protection for the neck according to the present invention;

FIG. 2 is a front view of paddings, contacted to the wearer's body, of the device of FIG. 1A;

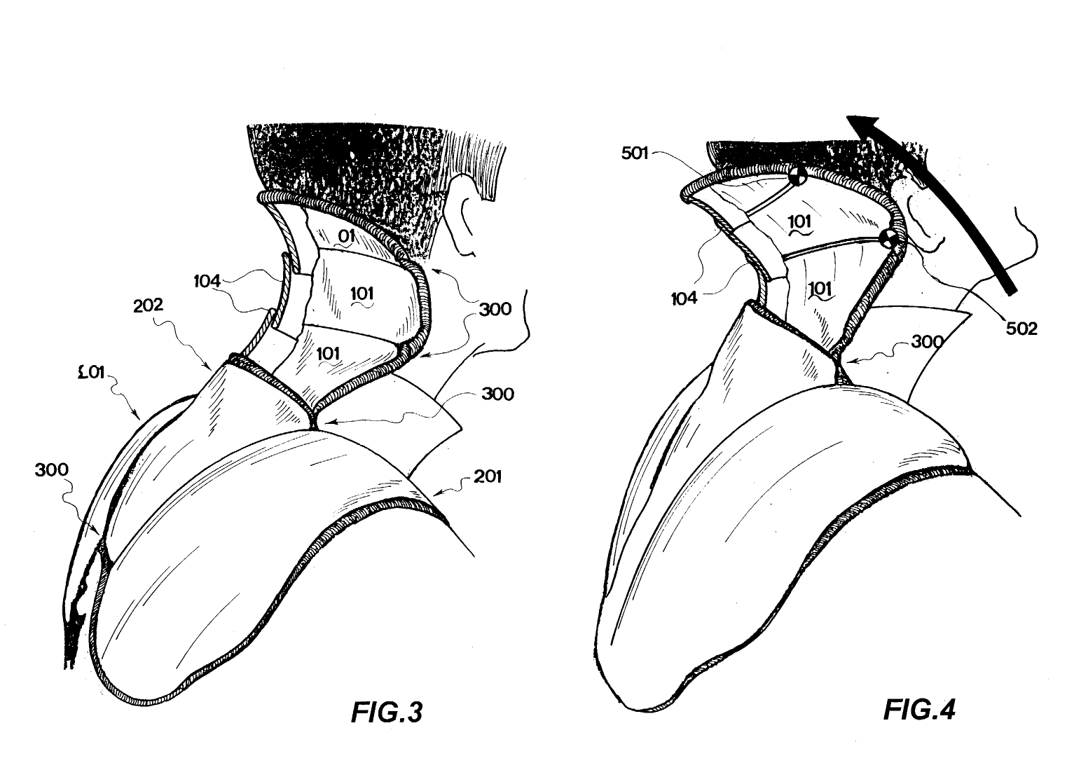

FIG. 3 is a partially sectional side view of the device according to the present invention once worn-on;

FIG. 4 is a sectional view illustrating the operation of the device according to the present invention; and

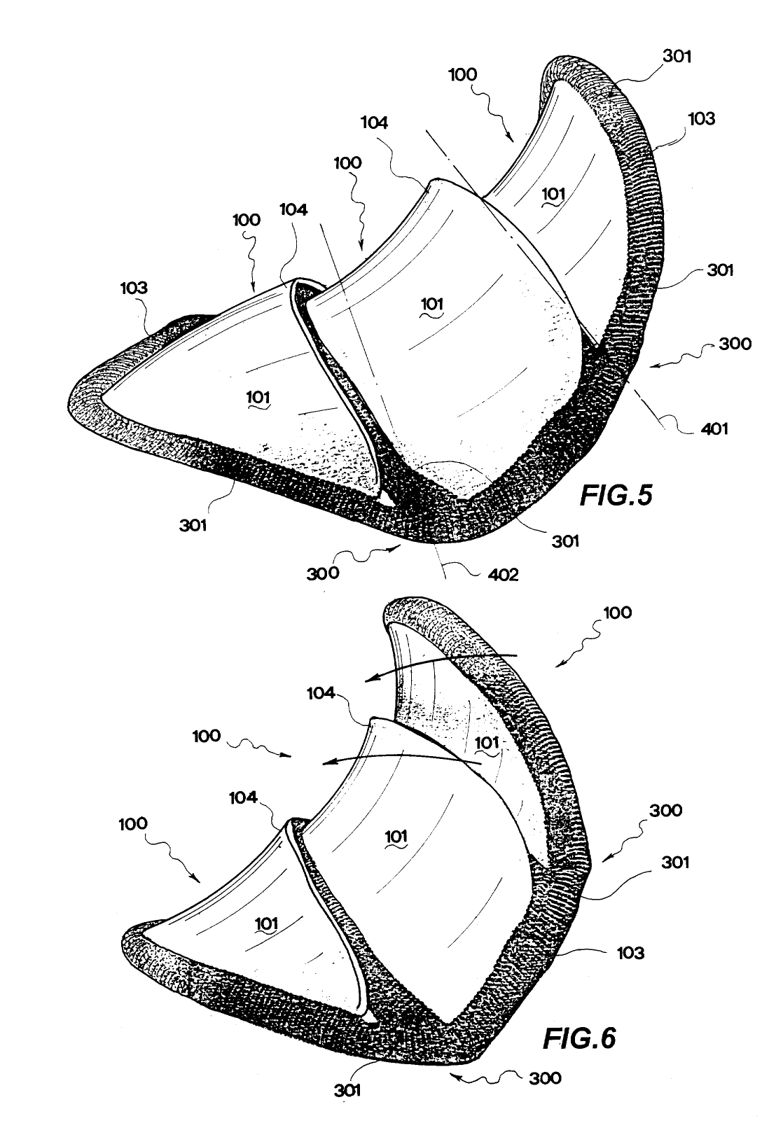

FIGS. 5 and 6 are side views illustrating connections deformable in two different operative positions, details of FIG. 1A.

Initially referring to FIG. 1A, a neck protective device according to the present invention comprises at least two protective elements 100, in the present embodiment three in number.

In particular, said protective elements comprise a central region 101 in a substantially rigid material.

Referring to FIGS. 2 and 3, the central region 101 is fastened to a padding 102 in a material that is substantially soft and therefore easily deformable.

According to a preferred embodiment, the padding 102 has a greater plane extension with respect to the regions 101, thus defining a peripheral portion 103, which will it also be in a substantially soft material.

Returning to FIG. 3, the central regions 101 and the paddings 102 have a sandwich-like arrangement, so that during the use of the device the padding 102 be arranged into contact with the user's body, whereas The central region 101 faces externally to the body.

Such a configuration allows an improved impact absorption and a high comfort to the user.

In fact, thus the neck is externally protected by the region 101 in a rigid material, preventing direct knocks against the user's body, whereas the padding, by being easily deformable, allows to at least partially absorb the energy of an impact.

Moreover, such a padding, being of soft material, feels particularly comfortable on the body.

The protective device according to the present invention further comprises supporting means, in particular, in the present embodiment, implemented by supporting elements 201 and 202, two at the shoulders and one onto the top portion of the spine.

Advantageously, also said supporting elements have features similar to the protective elements, so as to offer a protection also to the body parts respectively covered thereby.

Alternatively, the supporting means can be implemented by bands, or, always by way of example, be represented by a jacket connecting to the protective elements at the neck.

Basically, any means allowing to keep the protective elements at the user's neck may be used as supporting means.

According to the present embodiment, the supporting elements 201 and 202 are such as to be fastenable to a jacket, or other garment for the top part of the body.

Thus, the protective device according to the present invention is particularly susceptible of being integrated in a garment to be used during a sporting activity.

Moreover, the supporting elements can also comprise straps, not shown in the figure, apt to allow an improved fastening to the body.

As it will be detailed hereinafter, the device according to the present invention proves particularly effective; accordingly, an optimal protection could be attained even with a modest padding in the protective elements.

This feature proves particularly advantageous as it allows the wearability of the device below any jacket, without requiring it to have a special confectioning for the housing of the device.

Hence, the device can conveniently be inserted in a garment exclusively during a sporting activity, and removed afterwards.

In order to allow an easy wearability of the device, and the entailed removal thereof, the supporting elements have a curvature such as to adapt onto shoulders and shoulder blades.

This feature allows to alternatively wear the protective device merely below a jacket or other garment, with no need of additional fastening.

In fact, said shape allows to accurately position the device; the garment, as long as sufficiently clinging to the body, stably keeps such a position.

Concerning the operation of the device, returning to FIGS. 1A-1C, the protective elements 100 are connected thereamong by connecting means 300 allowing in particular the mutual rotation of the protective elements 100, and working as hinge.

According to a preferred embodiment, said means 300 is made with deformable elements 301.

Such deformable elements are fastened at side ends of the protective elements 100,

In particular, referring to FIG. 1B, said location enables said connecting means to allow the rotation of each protective element 100 with respect to respective axes of rotation 401, 402.

Moreover, as illustrated in FIGS. 1C and 2, the protective elements 100 have partially overlapped ends 104, so as to assure a continuous protection at the user's neck, avoiding the presence of openings that could allow a direct impact of the latter with any obstacles or with the ground itself.

The ends 104 will hereinafter be denoted by the adjective “overlapped”, indicating that they are overlapped to a further protective element. It is understood that the same definition indicates likewise the case in which it is had an inverted arrangement with the protective elements above the ends.

During the rotation allowed by said connecting means 300, the protective elements 100 always remain overlapped therebetween, thereby providing a continuous protection in all operative configurations.

Such a feature is implemented with a suitable arrangement of the axes of rotation 401 and 402 and the entailed dimension of the overlapped ends 104, which during the rotation of the protective elements 100 could be subjected to limited displacements, so as to keep below the respective protective elements.

However, the motion of the protective elements 100 is such as to assure the mobility required during the different sport activities and that the protections for the neck according to the known art cannot effectively assure.

Firstly, in fact, the mutual rotation of the protective elements 100 allows the backward tilting of the neck.

This motion is required, e.g., during motor bike activities in which the driver, owing to the driving posture, has to tilt back the neck, so as to raise the head and have a better vision of the road.

Or, another instance in which such a protection proves particularly useful is in case of skiing, above all at amateur level where in particular complete freedom of motion and comfort are required.

However, the backward tilting motion cannot be carried out by a mere rotation of the protective elements, as the presence of overlapped ends 104 hampers their free rotation.

In order to allow this motion, the connecting means 300 conveniently comprises deformable connections 301, allowing, besides a mutual rotation between the protective elements about the axes 401 and 402, also additional rotations with respect to different axes, orthogonal or tilted with respect to the preceding ones, as well as translation motions nearing thereto and/or away therefrom.

In the present embodiment the deformable connections are a strip-like element 301, fastened at the peripheral portion 103, basically joining the protective element at said side ends.

Moreover, according to the present embodiment, the strip-like element 301, made e.g. with a strip of fabric, extends along the entire perimeter of the protection, both in the external region and in that contacting the user's body, thereby enveloping together the protective elements, as well as the supporting elements, and implementing said connecting means 300.

It has to be noted that the main motion that should be carried out by the device is the mutual rotation between the protective elements along the axes 401 and 402.

During said motion, the strip-like element 301 bends, as highlighted in FIG. 4, yet this action not only implements the mutual rotation of the protective elements 100, but also those limited rotations with respect to different axes and/or limited translation motions nearing thereto and/or away therefrom.

By means of said limited motions, essentially allowing to arrange the relative position of the protective elements 100 the one with respect to the other, it is possible to attain the abovedescribed working configurations, so that the protective elements 100 always keep partially overlapped at the ends 104.

The strip-like elements are essentially made of a flexible material, e.g. of synthetic fabrics, and require no particular elasticity, since the deformability itself enables the abovedescribed combination of motions.

Moreover, the shape of the strip-like elements could differ from that of the mere plane strip, e.g. by preceding a central rib for higher resistance, in particular when heavier loads are envisaged, in case of particularly extreme activities.

Moreover, the connecting means 300 thus implemented allow limited lateral tilting motions of the head, by virtue of the flexibility of the deformable connections 301.

This feature proves particularly advantageous as it provides a high freedom of motion during sport activities, while always assuring a complete protection to the neck. Such effectiveness is not only related to the fact that also in this case it is kept the overlapping of the elements and the entailed continuity in the protection, but also to the fact that the device allows to limit the side tilting motions, thereby avoiding hazardous hyperextensions of the neck.

Always according to a preferred embodiment, during the motions of mutual rotation about the axes 401 and 402, the connecting means is made in a manner such as to make the protective elements 100 slide the one against the other.

In other words, since, as mentioned above, the shape of the protective elements and the positioning of the axes of rotation is such as not to allow a free rotation if not by slight deformations of the connecting means 300, during said rotations the overlapped ends 104 are not free to move, but their motion is hampered by the presence of the protective element thereabove.

Hence, while the rotation is possible, the motion occurs with the concomitant contact of the ends 104 against the protective elements 100, in particular at the peripheral portions 103 and at the paddings 102, This constructive feature becomes evident observing the location of respective centers of rotation 501 and 502 of the axes 401 and 402 in FIGS. 3 and 4. In fact, owing to this position the path undergone by the protective elements 100 interferes with the ends 104, causing the contact thereof,

Such a contact does not inhibit the rotation of the elements, due to the deformability of the connections 301, of the ends 104 and of the paddings 102, yet it limits the maximum range of the neck during the backward tilting.

In fact, with the increasing of the rotation between the protective elements, the resistance provided by the overlapped ends increases, as there increases the interference with the protective element to which it is overlapped. Hence, neither the deformable connections 301 nor peripheral portions 103 and paddings 102 are capable of deforming sufficiently to let the tilting motion of tile neck advance, beyond a certain angle.

Moreover, such a feature is shown in FIG. 4 in which it is described the operation of the protective device according to the present invention with the neck tilted backward.

The possibility of limiting such motions, with not completely inhibiting them, again proves particularly advantageous since a backward hyperextension of the neck, substantially the classic motion called “whiplash”, can have dire consequences as potentially prejudicial to the spine.

The protective device according to the present invention not only limits such motions, but also provides a dampening thereof.

In fact, also the overlapped ends preferably comprise a peripheral portion 103 in a deformable material that, during the rotation of the protective elements, is gradually deformed, optionally along with the padding 102, dissipating the energy that in case of impact tends to shift the neck backward.

Moreover, referring to FIG. 4 and always according to a preferred embodiment, the protective elements have a variable thickness, in particular the overlapped end 104 has a flattened shape.

Alternatively, the protective elements 100 can have a substantially wedge-shaped cross section.

Basically, such a flat shape can be implemented gradually making said wedge-shaped section.

Thus, besides the dampening effect created by the deformation of the peripheral portion 103 and of the padding 102, also the gradual wedging of a protective element with respect to that overlapped during their mutual rotation, at the flat zone, allows to exert a gradual resistance to motion.

Such a resistance can also be implemented by means of a particular configuration of the protective elements extending in a surface having a radius of curvature different with respect to that defined by the rotation of the overlapped ends 104. In the simplest case the protective elements can develop along a substantially plane surface; however, according to a preferred embodiment, they advantageously have a concavity such as to facilitate the wedging of the protective elements in the abovedescribed manner.

Moreover, the presence of a portion of protective element with a flattened shape allows also a greater stability of the protective device when worn-on, in particular when the neck is in a straight position.

Again referring to FIGS. 1A ad 1C, the above-described connecting means 300 is used also at the connection between the protective elements and the supporting elements, and between the supporting elements themselves.

Moreover, thus a further axis of rotation 403 is defined, between protective elements and the supporting element 201.

Thus, the same freedom of motion implemented for the neck is substantially implemented also at the portion joining neck and back and near the shoulder blades.

These features allow a high wearability, thereby making the protective device according to the present invention easily wearable under a jacket.

Moreover, they foster a high freedom of movements, jointly to the protection for back and shoulders, which proves particularly useful during sport activities.

Always by virtue of the use of the connecting means 300 also at the supporting elements, 201 and 202, the invention allows to provide a support for the neck, as the use of the structure formed by supporting elements and protective elements prevents the weight of the head from bearing entirely onto the neck.

In fact, the feature of limiting or anyhow containing neck motions proves particularly useful to provide a support to the neck also under non-traumatic conditions. Always by way of example, during motor biking practice the device according to the present invention can provide support to the neck and head during a normal ride, just because it limits the backward tilting of said body parts, advantageously providing the possibility of relaxing the neck muscles.

Likewise for back and shoulders, the protective device according to the present invention may be combined and optionally fastened with other protective elements, e.g. in correspondence of the breast, or of a helmet.

The present invention has hereto been described with reference to preferred embodiments thereof. It is understood that there may be other embodiments referable to the same inventive concept, all falling within the protective scope of the claims set forth hereinafter.

1-10. (canceled)

11. A neck protective device, comprising
at least two protective elements (100), said at least two protective elements (100) being partially overlapped and comprising respective overlapped ends (104), supporting means (200) of said at least two protective elements (100), and
respective connecting means (300) of said at least two protective elements (100), wherein said connecting means (300) is apt to allow the mutual rotation of said at least two protective elements (100), working as hinges along respective axes of rotation (401, 402), in a manner such that said at least two protective elements (100) slide the one against the other during said rotation and said overlapped ends (104) interfere with said at least two protective elements (100) limiting said rotation,
characterized in that said connecting means (300) comprises deformable connections (301) between said protective elements (100), so that the connecting means (300) allows, besides said rotation, also rotations according to axes different from said axes (401, 402) and/or limited translation motions nearing thereto and/or away therefrom, said connecting means (300) being fastened to a substantially soft peripheral portion (103) of said protective elements (100).

12. The protective device according to claim 11, wherein said at least two protective elements (100) have a flattened shape at said overlapped ends (104).

13. The protective device according to claim 11, wherein said protective elements (100) extend along a surface having a radius of curvature different with respect to that defined by the rotation of said overlapped ends (104).

14. The protective device according to claim 11, wherein said protective elements (100) have a substantially wedge-shaped cross section.

15. The protective device according to claim 11, wherein said at least two protective elements (100) comprise a substantially rigid central region (101) and a padding (102), assembled in a sandwich-like configuration.

16. The protective device according to claim 11, wherein said padding (102) has a greater plane extension with respect to said central region (101) defining said peripheral portion (103).

17. The protective device according to claim 11, wherein said connecting means (300) is fastened at side ends of said at least two protective elements (100).

18. The protective device according to claim 11, wherein said overlapped ends remain overlapped to said protective elements (100) during said motion of rotation about said axes (401, 402).

19. The protective device according claim 11, wherein said supporting means (200) comprises supporting elements (201, 202) suitable for the protection of shoulders and back.

20. The protective device according to claim 11, wherein said protective elements (100) are rotatably connected to said supporting means (200).
